(19) United States
(12) Patent Application Publication
(10) Pub. No.: US 2023/0330613 A1
(43) Pub. Date: Oct. 19, 2023

(54) METHANE PRODUCTION SYSTEM

(71) Applicant: INSTITUTE FOR ADVANCED ENGINEERING, Yongin-si (KR)

(72) Inventors: Young Don YOO, Seoul (KR); Su Hyun KIM, Suwon-si (KR); Min Hye SEO, Namyangju-si (KR)

(73) Assignee: INSTITUTE FOR ADVANCED ENGINEERING, Yongin-si (KR)

(21) Appl. No.: 18/213,919

(22) Filed: Jun. 26, 2023

Related U.S. Application Data
(63) Continuation of application No. PCT/KR2022/095009, filed on Jan. 11, 2022.

(30) Foreign Application Priority Data
Jan. 13, 2021 (KR) 10-2021-0004437

(51) Int. Cl. B01J 8/18; B01J 8/00

(52) U.S. Cl. CPC B01J 8/1827; B01J 8/002; B01J 8/1809; B01J 8/1836; B01J 2208/00061; B01J 2208/00115; B01J 2208/00539; B01J 2208/00769

(57) ABSTRACT

A methane production system includes: a raw material gas supply part configured to store and supply a raw material gas; a catalyst supply part configured to store and supply a catalyst; a methanation reaction part connected to the raw material gas supply part and the catalyst supply part and configured to generate a reaction gas by performing a methanation reaction using the raw material gas and the catalyst supplied from the raw material gas supply part and the catalyst supply part; a temperature measurement part connected to the methanation reaction part and configured to measure a temperature of the methanation reaction part; a temperature maintaining part connected to the raw material gas supply part; and a raw material gas injection part connected to the raw material gas supply part to receive the raw material gas from the raw material gas supply part.

### TECHNICAL FIELD

The present disclosure relates to a methane production system.

### BACKGROUND

As the climate change problem due to global warming has emerged, many countries around the world are conducting various studies to reduce the use of fossil fuels and increase the generation rate of new renewable energy in order to reduce greenhouse gas emissions.

However, since new renewable energy is greatly affected by weather and climate, the variability of power output is large, deteriorating the stability of a power system and causing a problem of idle power. An energy storage system (ESS) has been developed to overcome these limitations of the new renewable energy.

The ESS is a system that maximizes energy efficiency by storing the power generated from new renewable energy in a power grid when power demand is low and supplying the power when power demand is high. At this time, the ESS stores energy by a physical storage method such as pumped storage power generation, compressed air energy storage (CAES) or flywheel, or a chemical storage method such as lithium ion (Li-ion) battery, sodium sulfur battery (NaS) or VRB (Vanadium Redox Battery). However, in the case of the physical storage method, there is a problem that the initial facility investment cost is high, and in the case of the chemical storage method, there is a problem that the energy storage capacity and storage period are limited.

Due to these problems of the ESS, a Power-To-Gas (P2G) technique is being developed recently. The P2G technique is a technique that produces hydrogen by using electricity generated from new renewable energy or produces methane by subjecting hydrogen and carbon dioxide to a methanation reaction, and then stores the hydrogen or the methane in the form of fuel. In particular, when methane is produced using carbon dioxide, the effect of reducing carbon dioxide can be expected. Since carbon dioxide is a substance that accounts for about 90% of greenhouse gases, there is an advantage in that it is possible to reduce greenhouse gas emissions.

At this time, the methanation reaction in which hydrogen and carbon dioxide react to produce methane and water may be performed in a methanation reaction part such as a methanation reactor or the like. The methanation reaction is an exothermic reaction, and a transition metal-based catalyst is used for this methanation reaction.

However, when the temperature of the methanation reaction part rises to a high temperature of 900° C. or higher by the methanation reaction, the temperature of the methanation reaction part exceeds the temperature that a catalyst can withstand. Therefore, the sintering of the catalyst occurs, which reduces the activity of the catalyst and significantly reduces the methane conversion rate.

In addition, if a plurality of methanation reaction parts is connected and used in order to increase the methane conversion rate, the methane conversion rate is improved. However, the initial investment cost and operating cost increase due to the use of a plurality of reactors and auxiliary devices.

Therefore, there is a need for a methane production system capable of reducing initial investment costs and operating costs by simplifying a system configuration and maintaining the activity of a catalyst to improve a methane conversion rate.

### SUMMARY

The embodiments of the present disclosure have been conceived to solve the aforementioned problems of the related art, and provide a methane production system capable of suppressing the occurrence of hot spots and uniformly controlling the temperature of a reactor with a simple system configuration, maintaining the activity of a catalyst, improving the methane conversion rate through the enhanced contact between a raw material gas and a catalyst, and consequently reducing the initial investment and operating costs.

According to one aspect of the present disclosure, there may be provided a methane production system, including: a raw material gas supply part configured to store and supply a raw material gas; a catalyst supply part configured to store and supply a catalyst; a methanation reaction part connected to the raw material gas supply part and the catalyst supply part and configured to generate a reaction gas by performing a methanation reaction using the raw material gas and the catalyst supplied from the raw material gas supply part and the catalyst supply part; a temperature measurement part connected to the methanation reaction part and configured to measure a temperature of the methanation reaction part; a temperature maintaining part connected to the raw material gas supply part and configured to maintain the temperature of the methanation reaction part at a predetermined temperature based on the temperature of the methanation reaction part measured by the temperature measurement part; and a raw material gas injection part connected to the raw material gas supply part to receive the raw material gas from the raw material gas supply part and configured to inject the raw material gas into the methanation reaction part at a flow rate higher than a flow rate at which the raw material gas is supplied from the raw material gas supply part.

In addition, the temperature maintaining part may include a cooling medium supply part configured to store and supply a cooling medium, and a heat exchanger connected to the cooling medium supply part and configured to exchange heat between the cooling medium supplied from the cooling medium supply part and the reaction heat generated through the methanation reaction, and the temperature of the cooling medium supplied from the cooling medium supply part to the heat exchanger may be determined according to the temperature of the methanation reaction part measured by the temperature measurement part.

In addition, the methanation reaction part may include a reaction body configured to provide a space in which the methanation reaction between the raw material gas and the catalyst is performed, and the heat exchanger may be provided inside the reaction body and installed to be spaced apart from an inner surface of the reaction body.

In addition, an upper flow path through which the raw material gas flows upward may be formed in the heat exchanger, and a lower flow path through which the raw material gas flows downward may be formed between the inner surface of the reaction body and the heat exchanger.

In addition, the system may further include: a changing part provided inside the reaction body and arranged above the heat exchanger so that the raw material gas flowing upward along the upper flow path enters the lower flow path after colliding with the changing part.

In addition, an upper end of the raw material gas injection part may be disposed at a higher position than a lower end of the heat exchanger so that the raw material gas flowing along the lower flow path enters the upper flow path due to a pressure change between the upper end of the raw material gas injection part and the lower end of the heat exchanger.

In addition, the methanation reaction part may further include a catalyst supply port connected at an end to the reaction body and configured to receive the catalyst from the catalyst supply part, a catalyst discharge port connected at an end to the reaction body and configured to discharge the catalyst used in the methanation reaction, a cooling medium supply port having one end connected to the heat exchanger and the other end protruding outward from the reaction body, and configured to receive the cooling medium from the cooling medium supply part, and a cooling medium recovery port having one end connected to the heat exchanger and the other end protruding outward from the reaction body, and configured to recover the cooling medium, which has absorbed the reaction heat generated through the methanation reaction, to the cooling medium supply part.

In addition, the system may further include: a reaction gas processing part connected to the methanation reaction part and configured to post-process the reaction gas supplied from the methanation reaction part; a reaction gas analysis part connected to the reaction gas processing part and configured to analyze the post-processed reaction gas received from the reaction gas processing part; and a reaction gas recirculation part connected between the reaction gas analysis part and the raw material gas supply part, and configured to supply at least a part of the post-processed reaction gas to the raw material gas supply part according to the analysis value of the reaction gas analysis part.

In addition, the system may further include: a catalyst discharge part connected to the methanation reaction part, wherein the catalyst discharge part may be configured to discharge the catalyst from the methanation reaction part according to the supply amount of the catalyst supplied to the methanation reaction part through the catalyst supply part.

In addition, the system may further include: a control part configured to control at least one of the raw material gas supply part, the catalyst supply part, the methanation reaction part, the temperature measurement part, the temperature maintaining part, the raw material gas injection part, the changing part, the reaction gas processing part, the reaction gas analysis part, the reaction gas recirculation part and the catalyst discharge part.

In addition, the temperature maintaining part may further include a cooling medium pressure regulation line connected to the cooling medium supply part, a cooling medium pressure regulation valve installed in the cooling medium pressure regulation line, and a cooling medium recovery line arranged to connect the cooling medium recovery port and the cooling medium supply part, and configured to recover the cooling medium, which has absorbed the reaction heat of the methanation reaction, to the cooling medium supply part, and the control part may be configured to control the temperature of the cooling medium supplied to the heat exchanger by controlling the opening and closing of the cooling medium pressure regulation valve according to the temperature of the methanation reaction part measured by the temperature measurement part.

In addition, the catalyst supply part may include a catalyst storage tank configured to store the catalyst, a catalyst supply line arranged to connect the catalyst storage tank and the catalyst supply port and configured to supply the catalyst to the reaction body, and a catalyst supply valve installed in the catalyst supply line, and the control part may be configured to control to adjust the amount of catalyst supplied to the reaction body by controlling the opening and closing of the catalyst supply valve according to the analysis value of the reaction gas analyzed by the reaction gas analysis part.

In addition, the catalyst discharge part may include a catalyst discharge line connected to the catalyst discharge port, and a catalyst discharge valve installed in the catalyst discharge line, and the control part may be configured to adjust the amount of catalyst discharged from the reaction body by controlling the opening and closing of the catalyst discharge valve according to the amount of catalyst supplied to the reaction body by the catalyst supply part.

In addition, the reaction gas processing part may include a reaction gas discharge line connected to the reaction body, a catalyst recirculation part connected to the reaction gas discharge line and configured to receive the reaction gas from the reaction body, separate the catalyst contained in the reaction gas and supply the separated catalyst to the reaction body, a cooler connected to the catalyst recirculation part and configured to cool the catalyst-separated reaction gas received from the catalyst recirculation part, a first connection line configured to connect the catalyst recirculation part and the cooler, a gas-liquid separator configured to separate the reaction gas received from the cooler into a gas product and a liquid product, and a second connection line configured to connect the cooler and the gas-liquid separator.

In addition, the methanation reaction part may further include a catalyst re-supply port connected at an end to the reaction body and configured to receive the catalyst separated through the catalyst recirculation part.

In addition, the catalyst recirculation part may include a dust collector connected to the reaction gas discharge line and configured to separate the catalyst contained in the reaction gas, and a catalyst recirculation line configured to connect the dust collector and the catalyst re-supply port.

In addition, the reaction gas processing part may further include a first discharge line connected to the gas-liquid separator and configured to discharge one of the gas product and the liquid product separated through the gas-liquid separator, a second discharge line connected to the gas-liquid separator and configured to discharge the other of the gas product and the liquid product separated through the gas-liquid separator, and a process pressure regulation valve installed in the first discharge line and provided behind the reaction gas analysis part.

In addition, the reaction gas analysis part may be installed in the first discharge line and configured to analyze the composition of the gas product separated through the gas-liquid separator.

In addition, the reaction gas recirculation part may include a first reaction gas recirculation line branched from at least one of the first discharge line and the second discharge line, a compressor installed in the first reaction gas recirculation line, a reaction gas recirculation valve installed in the first reaction gas recirculation line and disposed in front of the compressor, and a second reaction gas recirculation line connected between the compressor and the raw material gas supply part, and the control part may be configured to supply at least a part of the gas product separated through the gas-liquid separator to the raw material gas supply part by controlling the opening and closing of the reaction gas recirculation valve according to the analysis value of the reaction gas analyzed by the reaction gas analysis part.

According to the embodiments of the present disclosure, it is possible to maintain the activity of a catalyst with a simple system configuration, improve the methane conversion rate, and consequently reduce the initial investment and operating costs.

### BRIEF DESCRIPTION OF THE DRAWINGS

FIG. 1 is a conceptual diagram showing a methane production system according to an embodiment of the present disclosure.

FIG. 2 is a diagram showing a methanation reaction part of the methane production system of FIG. 1.

FIG. 3 is a control block diagram of the methane production system of FIG. 1.

### DETAILED DESCRIPTION

Hereinafter, specific embodiments for implementing the spirit of the present disclosure will be described in detail with reference to the drawings.

In addition, in describing the present disclosure, if it is determined that a detailed description of a related known configuration or function may obscure the subject matter of the present disclosure, the detailed description thereof will be omitted.

In addition, when one component is referred to as being ‘connected to’, ‘supported by’ or ‘in contact with’ another component, it should be understood that one component may be directly connected to, supported by or in contact with another component and a further component may exist between one component and another component.

The terms used in the subject specification are only used to describe specific embodiments, and are not intended to limit the present disclosure. Singular expressions include plural expressions unless the context clearly indicates otherwise.

In addition, in the subject specification, expressions such as one side, the other side, and the like are defined with reference to the illustration in the drawings. It should be noted that if the direction of the corresponding object is changed, the object may be expressed differently. For the same reason, some components in the accompanying drawings are exaggerated, omitted, or schematically illustrated. The size of each component does not thoroughly reflect the actual size.

In addition, the terms including ordinal numbers such as first and second may be used to describe various components, but the corresponding components are not limited by such terms. These terms are only used for the purpose of distinguishing one component from another.

The meaning of “comprise” or “include” as used in the specification specifies a specific characteristic, region, integer, step, operation, element and/or component, and does not exclude the existence or addition of other specific characteristic, region, integer, step, operation, element, component and/or group.

Hereinafter, the detailed configuration of a methane production system according to an embodiment of the present disclosure will be described with reference to the drawings.

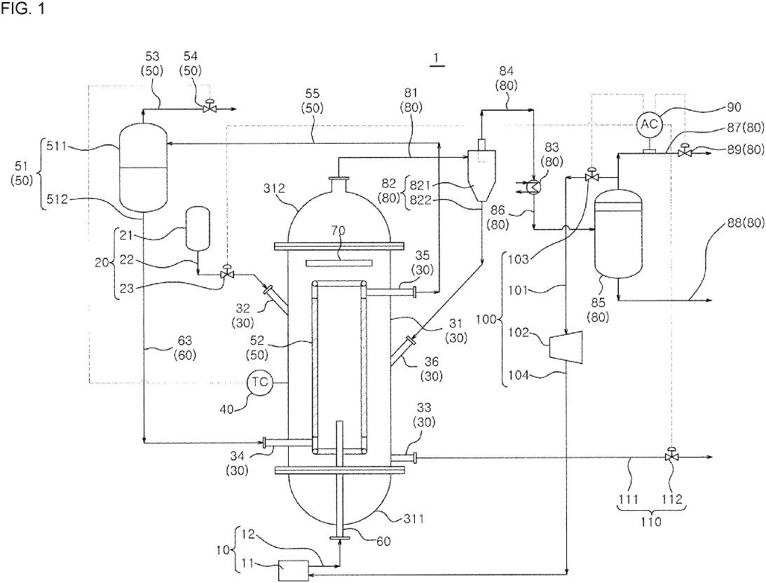

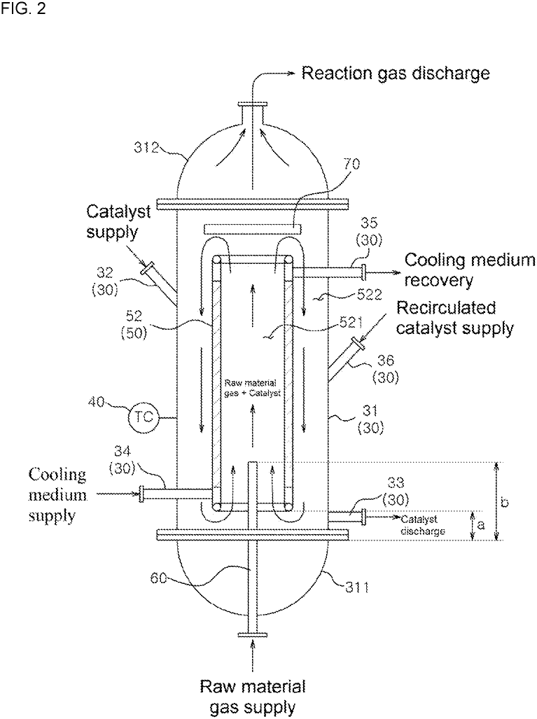

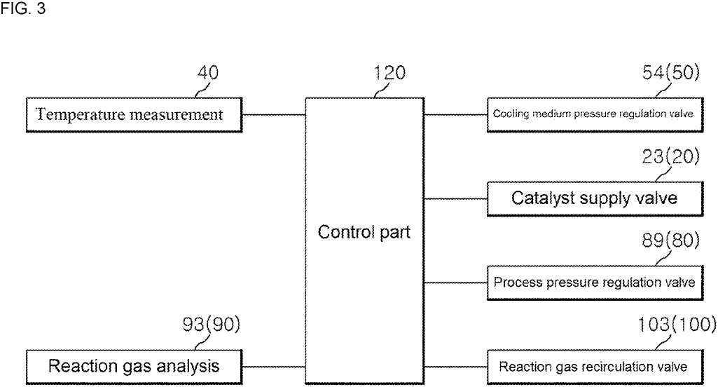

Referring to FIGS. 1 to 3, the methane production system 1 according to an embodiment of the present disclosure may include a raw material gas supply part 10, a catalyst supply part 20, a methanation reaction part 30, a temperature measurement part 40, a temperature maintaining part 50, a raw material gas injection part 60, a changing part 70, a reaction gas processing part 80, a reaction gas analysis part 90, a reaction gas recirculation part 100, a catalyst discharge part 110 and a control part 120.

The precursor gas supply part 10 may store a raw material gas and may supply the raw material gas to the methanation reaction part 30. To this end, the raw material gas supply part 10 may include a raw material gas storage tank 11 and a raw material gas supply line 12.

The raw material gas storage tank 11 may provide a storage space capable of storing the raw material gas. For example, the raw material gas stored in the raw material gas storage tank 11 may be a gas containing carbon dioxide and hydrogen in a molar ratio of 1:4.

At this time, the raw material gas stored in the raw material gas storage tank 11 may be heated to a temperature higher than the activation temperature of the catalyst stored in the catalyst supply part 20. The raw material gas heated in this way may be supplied to the raw material gas injection part 60 through a raw material gas supply line 12 connected to the raw material gas storage tank 11, and then may be supplied to the methanation reaction part 30 through the raw material gas injection part 60. When the raw material gas heated to a temperature higher than the activation temperature of the catalyst is supplied to the methanation reaction part 30, a methanation reaction may be performed while as the raw material gas flows in the methanation reaction part 30 together with the catalyst supplied to the methanation reaction part 30. Details thereof will be described later.

Meanwhile, the reaction gas discharged from the methanation reaction part 30 and then passing through the reaction gas processing part 80 and the reaction gas analysis part 90 may be supplied to the raw material gas storage tank 11 through the reaction gas recirculation part 100. The reaction gas supplied to the raw material gas storage tank 11 through the reaction gas recirculation part 100 in this way may be mixed with the raw material gas stored in the raw material gas storage tank 11, and then may be supplied to the methanation reaction part 30 through the raw material gas injection part 60.

The catalyst supply part 20 may store a catalyst and may supply the catalyst to the methanation reaction part 30. To this end, the catalyst supply part 20 may include a catalyst storage tank 21 configured to store the catalyst, a catalyst supply line 22 configured to connect the catalyst storage tank 21 and the catalyst supply port 32 of the methanation reaction part 30, which will be described later, and supply the catalyst to the reaction body 31 of the methanation reaction part 30, which will be described later, and a catalyst supply valve 23 installed in the catalyst supply line 22.

The catalyst storage tank 21 is a part that provides a storage space capable of storing a catalyst. For example, the catalyst stored in the catalyst storage tank 21 may be a transition metal catalyst such as nickel (Ni), cobalt (Co), ruthenium (Ru), or the like. In this case, the catalyst may have a powder form and may have a particle size of about 5 µm to about 100 µm.

At this time, the catalyst in the catalyst storage tank 21 may be supplied to the reaction body 31 through the catalyst supply line 22 in response to the opening and closing of the catalyst supply valve 23. The opening and closing of the catalyst supply valve 23 may be controlled by the control part 120. For example, the opening and closing of the catalyst supply valve 23 may be controlled according to the analysis value analyzed by the reaction gas analysis part 90. In addition, the opening and closing of the catalyst supply valve 23 may be controlled in conjunction with the opening and closing of the catalyst discharge valve 112 of the catalyst discharge part 110, which will be described later. Details thereof will be described later.

The methanation reaction part 30 may generate a reaction gas by performing a methanation reaction using the raw material gas and the catalyst supplied from the raw material gas supply part 10 and the catalyst supply part 20, respectively. To this end, the methanation reaction part 30 may be connected to the raw material gas supply part 10 and the catalyst supply part 20.

In this case, the methanation reaction part 30 may include a reaction body 31, a catalyst supply port 32, a catalyst discharge port 33, a cooling medium supply port 34, a cooling medium recovery port 35, and a catalyst re-supply port 36.

The reaction body 31 may provide a space in which the methanation reaction of the raw material gas and the catalyst can be performed. In this case, the reaction body 31 may be provided as a tubular reactor with both ends thereof opened. A lower cover 311 may be coupled to the lower end of the reaction body 31, and an upper cover 312 may be coupled to the upper end of the reaction body 31. The raw material gas injection part 60 may be coupled to the lower cover 311. At least a portion of the raw material gas injection part 60 coupled to the lower cover 311 may be disposed inside the reaction body 31. The reaction gas discharge line 81 of the reaction gas processing part 80, which will be described later, may be coupled to the upper cover 312.

The catalyst supply port 32 may receive the catalyst from the catalyst supply part 20. To this end, the end portion of the catalyst supply port 32 may be connected to the reaction body 31, and the catalyst supply line 22 may be connected to the catalyst supply port 32.

The catalyst discharge port 33 may discharge the catalyst used in the methanation reaction. To this end, the end portion of the catalyst discharge port 33 may be connected to the reaction body 31, and the catalyst discharge line 111 of the catalyst discharge part 110, which will be described later, may be connected to the catalyst discharge port 33.

The cooling medium supply port 34 may receive a cooling medium from the cooling medium supply part 51 of the temperature maintaining part 50, which will be described later. To this end, one end of the cooling medium supply port 34 may be connected to the heat exchanger 52 of the temperature maintaining part 50, which will be described later, and the other end of the cooling medium supply port 34 may protrude outward from the reaction body 31. The cooling medium supply line 512 of the cooling medium supply part 51, which will be described below, may be connected to the other end of the cooling medium supply port 34 protruding outward from the reaction body 31.

The cooling medium recovery port 35 may recover the cooling medium that has absorbed reaction heat generated in the methanation reaction to the cooling medium supply part 51. To this end, one end of the cooling medium recovery port 35 may be connected to the heat exchanger 52, and the other end of the cooling medium recovery port 35 may protrude outward from the reaction body 31. The cooling medium recovery line 55 of the temperature maintaining part 50, which will be described below, may be connected to the other end of the cooling medium recovery port 35 protruding outward from the reaction body 31.

The catalyst re-supply port 36 may receive the separated catalyst through the catalyst recirculation part 82 of the reaction gas processing part 80, which will be described later. To this end, the end portion of the catalyst re-supply port 36 may be connected to the reaction body 31. The catalyst recirculation line 822 of the catalyst recirculation part 82 of the reaction gas processing part 80, which will be described later, may be connected to the catalyst re-supply port 36.

The temperature measurement part 40 may measure the temperature of the methanation reaction part 30. To this end, the temperature measurement part 40 may be connected to the methanation reaction part 30 and may be provided as a temperature sensor, for example. The temperature value of the methanation reaction part 30 measured by the temperature measurement part 40 may be transmitted to the temperature maintaining part 50 and may be used to maintain the temperature of the methanation reaction part 30 at a preset temperature. Details thereof will be described later.

The temperature maintaining part 50 may maintain the temperature of the methanation reaction part 30 at a predetermined temperature based on the temperature value of the methanation reaction part 30 measured by the temperature measurement part 40. To this end, the temperature maintaining part 50 may be connected to the methanation reaction part 30.

The temperature maintaining part 50 may include a cooling medium supply part 51 configured to store and supply a cooling medium, a heat exchanger 52 connected to the cooling medium supply part 51 and configured to exchange heat between the cooling medium supplied from the cooling medium supply part 51 and the reaction heat generated through the methanation reaction, a cooling medium pressure regulation line 53 connected to the cooling medium supply part 51, a cooling medium pressure regulation valve 54 installed in the cooling medium pressure regulation line 53, and a cooling medium recovery line 55 configured to connect the cooling medium recovery port 35 and the cooling medium supply part 51 and configured to recover the cooling medium that has absorbed the reaction heat of the methanation reaction to the cooling medium supply part 51.

The cooling medium supply part 51 may include a cooling medium storage tank 511 configured to store the cooling medium, and a cooling medium supply line 512 connected to the cooling medium storage tank 511.

The cooling medium storage tank 511 may provide a space for storing a cooling medium. In this case, the cooling medium may have a liquid state, and may be, for example, saturated water. This cooling medium may absorb the reaction heat generated by the methanation reaction in the methanation reaction part 30 that affects the activity of the catalyst in the methanation reaction part 30.

In this case, the cooling medium in the cooling medium storage tank 511 may be supplied to the heat exchanger 52 through the cooling medium supply line 512. The cooling medium, for example, saturated water, supplied to the heat exchanger 52 may be evaporated by absorbing the reaction heat of the methanation reaction in the methanation reaction part 30, thereby having a gaseous state. For example, the cooling medium that has absorbed the reaction heat of the methanation reaction may be saturated steam. Such saturated steam may be supplied back to the cooling medium storage tank 511 through the cooling medium recovery line 55. The saturated steam supplied back to the cooling medium storage tank 511 may be used to adjust the temperature of the saturated water stored in the cooling medium storage tank 511. At this time, the opening and closing of the cooling medium pressure regulation valve 54 may be controlled by the control part 120 based on the temperature of the methanation reaction part 30 measured by the temperature measurement part 40. As the saturated steam is selectively discharged through the cooling medium pressure regulation line 53 in response to the opening and closing of the cooling medium pressure regulation valve 54, the temperature of the saturated water stored in the cooling medium storage tank 511 may be adjusted.

The heat exchanger 52 may cause the reaction heat of the methanation reaction in the methanation reaction part 30 to be absorbed into the cooling medium supplied from the cooling medium supply part 51, thereby maintaining the temperature of the methanation reaction part 30 to a preset temperature.

In addition, the heat exchanger 52 may form a flow path through which the raw material gas injected from the raw material gas injection part 60 can flow. To this end, the heat exchanger 52 may be provided inside the reaction body 31.

For example, the heat exchanger 52 may be provided in a tubular shape as a whole with both ends thereof opened. In addition, the heat exchanger 52 is arranged in a circular shape and may include a tube surrounded by a diaphragm. Accordingly, an upper flow path 521 through which the raw material gas flows upward may be formed in the heat exchanger 52. In this case, the heat exchanger 52 may be installed so as to be spaced apart from the inner surface of the reaction body 31. A lower flow path 522 through which the raw material gas flows downward may be formed between the inner surface of the reaction body 31 and the heat exchanger 52.

In this case, the methanation reaction may be performed between the raw material gas flowing upward along the upper flow path 521 and the catalyst. When the raw material gas flowing upward along the upper flow path 521 collides with the changing part 70 provided above the heat exchanger 52, the flow direction of the raw material gas may be changed from an upward direction to a downward direction. The raw material gas whose flow direction is changed to the downward direction may enter the lower flow path 522. The methanation reaction may be performed once again between the raw material gas flowing downward along the lower flow path 522 and the catalyst.

Meanwhile, the upper end of the raw material gas injection part 60 may be disposed at a higher position than the lower end of the heat exchanger 52. For example, the distance (a) between the bottom surface of the reaction body 31 and the lower end of the heat exchanger 52 may be smaller than the distance (b) between the bottom surface of the reaction body 31 and the upper end of the raw material gas injection part 60. Accordingly, a change in the pressure of the raw material gas may occur between the upper end of the raw material gas injection part 60 and the lower end of the heat exchanger 52, and a pressure drop may occur below the raw material gas injection part 60. Due to this, the raw material gas flowing along the lower flow path 522 may enter the upper flow path 521. Accordingly, the raw material gas may flow while circulating through the upper flow path 521 and the lower flow path 522. The raw material gas flowing while circulating through the upper flow path 521 and the lower flow path 522 is mixed with and contacted with the catalyst present in the upper flow path 521 and the lower flow path 522, so that methanation reactions are performed in both the upper flow path 521 and the lower flow path 522.

The raw material gas injection part 60 may be connected to the raw material gas supply part 10 to receive the raw material gas from the raw material gas supply part 10, and may inject the raw material gas toward the methanation reaction part 30 at a flow rate higher than the flow rate at which the raw material gas is supplied from the raw material gas supply part 10.

At this time, the raw material gas and the catalyst may be mixed in the entire region of the methanation reaction part 30 depending on the injection flow rate of the raw material gas injected through the raw material gas injection part 60. Thus, the methanation reaction may be performed over the entire region of the methanation reaction part 30.

Meanwhile, the raw material gas injection part 60 may be coupled to the lower cover 311 connected to the lower end of the reaction body 31. For example, the raw material gas injection part 60 may be an injection nozzle having a shape extending in one direction. In this case, at least a portion of the raw material gas injection part 60 may be disposed inside the reaction body 31. Accordingly, the raw material gas may be injected toward the center of the heat exchanger 52, for example, the upper flow path 521.

The changing part 70 may change the flow direction of the raw material gas flowing in the reaction body 31. To this end, the changing part 70 may be provided inside the reaction body 31 and may be disposed above the heat exchanger 52. In this case, the changing part 70 may block the flow of the raw material gas flowing upward along the upper flow path 521 inside the reaction body 31 to change the flow direction of the raw material gas from the upward direction to the downward direction.

In this case, the circumferential surface of the changing part 70 may be spaced apart from the inner surface of the reaction body 31. The reaction gas generated through the methanation reaction in the reaction body 31 may pass through the separation space between the circumferential surface of the changing part 70 and the inner surface of the reaction body 31. A catalyst may be included in the reaction gas passing through the space between the circumferential surface of the changing part 70 and the inner surface of the reaction body 31. The catalyst may be separated through the reaction gas processing part 80.

The reaction gas processing part 80 may be connected to the methanation reaction part 30 to receive the reaction gas from the methanation reaction part 30 and post-process the reaction gas. To this end, the reaction gas processing part 80 includes a reaction gas discharge line 81 connected to the reaction body 31, a catalyst recirculation part 82 connected to the reaction gas discharge line 81 and configured to receive the reaction gas from the reaction body 31, separate the catalyst contained in the reaction gas and supply the separated catalyst to the reaction body 31, a cooler 83 connected to the catalyst recirculation part 82 and configured to cool the catalyst-separated reaction gas received from the catalyst recirculation part 82, a first connection line 84 configured to connect the catalyst recirculation part 82 and the cooler 83, a gas-liquid separator 85 configured to separate the reaction gas received from the cooler 83 into a gas product and a liquid product, and a second connection line 86 configured to connect the cooler 83 and the gas-liquid separator 85. As used herein, the term “post-processing process” performed in the reaction gas processing part 80 refers to a process of separating the catalyst from the reaction gas discharged from the reaction body 31, a process of cooling the catalyst-separated reaction gas through the cooler 83, and a process of gas-liquid separating the reaction gas cooled in the cooler 83 through the gas-liquid separator 85. For example, the gas product separated in the gas-liquid separator 85 may be a methane gas, and the liquid product separated in the gas-liquid separator 85 may be water.

In this case, the catalyst recirculation part 82 may include a dust collector 821 connected to the reaction gas discharge line 81 and configured to separate the catalyst contained in the reaction gas, and a catalyst recirculation line 822 configured to connect the dust collector 821 and the catalyst re-supply port 36. The catalyst separated through the dust collector 821 may be supplied to the reaction body 31 through the catalyst recirculation line 822, and the reaction gas from which the catalyst is separated through the dust collector 821 may be supplied to the cooler 83 through the first connection line 84.

In addition, the reaction gas processing part 80 may further include a first discharge line 87 connected to the gas-liquid separator 85 and configured to discharge one of the gas product and the liquid product separated through the gas-liquid separator 85, a second discharge line 88 connected to the gas-liquid separator 85 and configured to discharge the other of the gas product and the liquid product separated through the gas-liquid separator 85, and a process pressure regulation valve 89 installed in the first discharge line 87 and provided behind the reaction gas analysis part 90.

The reaction gas analysis part 90 may be connected to the reaction gas processing part 80 and may receive the post-processed reaction gas from the reaction gas processing part 80 and analyze the post-processed reaction gas. Specifically, the reaction gas analysis part 90 may be installed in the first discharge line 87 and may analyze the composition of the gas product separated through the gas-liquid separator 85.

Meanwhile, an analysis value, for example, methane conversion rate, analyzed by the reaction gas analysis part 90 may be transmitted to the control part 120. The control part 120 may use the methane conversion rate analyzed by the reaction gas analysis part 90 to control the catalyst supply valve 23, the process pressure regulation valve 88, and the reaction gas recirculation valve 103 of the reaction gas recirculation part 100, which will be described later. For example, the control part 120 may compare the methane conversion rate analyzed by the reaction gas analysis part 90 with a preset methane conversion rate. If the methane conversion rate analyzed by the reaction gas analysis part 90 is lower than the preset methane conversion rate, the control part 120 may open the catalyst supply valve 23 so that the catalyst can be additionally supplied to the reaction body 31.

Further, the control part 120 may compare the methane conversion rate analyzed by the reaction gas analysis part 90 with a preset methane conversion rate. If the methane conversion rate analyzed by the reaction gas analysis part 90 exceeds the preset methane conversion rate, the control part 120 may control the process pressure regulation valve 88 so as to be opened.

In addition, the control part 120 may compare the methane conversion rate analyzed by the reaction gas analysis part 90 with a preset methane conversion rate. If the methane conversion rate analyzed by the reaction gas analysis part 90 is lower than the preset methane conversion rate, the control part 120 may control the reaction gas recirculation valve 103 so as to be opened. Details thereof will be described later.

The reaction gas recirculation part 100 may supply at least a part of the post-processed reaction gas to the raw material gas supply part 10 according to the analysis value of the reaction gas analysis part 90. To this end, the reaction gas recirculation part 100 may be connected between the reaction gas analysis part 90 and the raw material gas supply part 10.

Meanwhile, the reaction gas recirculation part 100 may include a first reaction gas recirculation line 101 branched from at least one of the first discharge line 87 and the second discharge line 88, a compressor 102 installed in the first reaction gas recirculation line 101, a reaction gas recirculation valve 103 installed in the first reaction gas recirculation line 101 and disposed in front of the compressor 102, and a second reaction gas recirculation line 104 connected between the compressor 102 and the raw material gas supply part 10.

In this case, the opening and closing of the reaction gas recirculation valve 103 may be controlled by the control part 120. The control part 120 may compare the methane conversion rate analyzed by the reaction gas analysis part 90 with a preset methane conversion rate. If the methane conversion rate analyzed by the reaction gas analysis part 90 is lower than the preset methane conversion rate, the control part 120 may open the reaction gas recirculation valve 103 so that the gas product separated by the gas-liquid separator 85 can be supplied to the raw material gas storage tank 11 through the compressor 102. The gas product supplied to the raw material gas storage tank 11 may be mixed with the raw material gas and may be supplied to the reaction body 31 through the raw material gas injection part 60.

In the present embodiment, the case where the reaction gas recirculation valve 103 is provided in front of the compressor 102 and the reaction gas is recirculated to the reaction body 31 by the reaction gas recirculation valve 103 has been described by way example. However, the present disclosure is not limited thereto. If necessary, the reaction gas recirculation valve 103 may be omitted. In this case, the reaction gas may be supplied to the raw material gas storage tank 11 by the compression and discharge operations of the compressor 102.

The catalyst discharge part 110 may discharge the catalyst from the methanation reaction part 30 according to the supply amount of the catalyst supplied to the methanation reaction part 30 through the catalyst supply part 20. To this end, the catalyst discharge part 110 may include a catalyst discharge line 111 connected to the catalyst discharge port 33 and a catalyst discharge valve 112 installed in the catalyst discharge line 111.

Meanwhile, the amount of catalyst discharged from the reaction body 31 may be determined by opening and closing the catalyst discharge valve 112. At this time, the opening and closing of the catalyst discharge valve 112 may be controlled by the control part 120. The control part 120 may control the amount of catalyst discharged from the reaction body 31 by controlling the opening and closing of the catalyst discharge valve 112 according to the amount of catalyst supplied to the reaction body 31 by the catalyst supply part 20.

According to the methane production system 1 according to the embodiment of the present disclosure described above, the temperature of the methanation reaction part 30 where the terminal gas and the catalyst are supplied to perform the methanation reaction is controlled to be maintained at a preset temperature. As a result, the occurrence of hot spots is suppressed and the temperature of the methanation reaction part 30 is uniformly controlled, so that the activity of the catalyst in the methanation reaction part 30 can be maintained and the lifespan of the catalyst can be prolonged.

In addition, the raw material gas is injected into the methanation reaction part 30 at a predetermined flow rate through the raw material gas injection part 60, and the raw material gas in the methanation reaction part 30 circulates and flows along the upper flow path and the lower flow path at a predetermined flow rate, whereby the contact performance and contact time of the raw material gas and the catalyst can be increased to improve the methane conversion rate.

As a result, the methane conversion rate can be improved with a simple system configuration, which makes it possible to reduce the initial investment cost and operating cost of the methane production system 1.

In addition, the methane conversion rate of the reaction gas, which is a reaction product of the methanation reaction in the methanation reaction part 30, can be analyzed through the reaction gas analysis part 90. If the analysis value for example, the methane conversion rate, which is analyzed by the reaction gas analysis part 90, is lower than a preset methane conversion rate value, the catalyst is additionally supplied to the methanation reaction part 30 so that the methanation reaction can be additionally performed in the methanation reaction part 30. This can improve the energy efficiency. At this time, the catalyst is discharged from the methanation reaction part 30 as much as the amount of catalyst supplied to the methanation reaction part 30 through the catalyst supply part 20. Thus, the catalyst amount in the methanation reaction part 30 can be kept constant.

In addition, if the analysis value, for example, the methane conversion rate, which is analyzed by the reaction gas analysis part 90, is lower than the preset methane conversion rate, the reaction gas is supplied to the methanation reaction part 30 again so that the methanation reaction can be additionally performed in the methanation reaction part 30. This can improve the energy efficiency.

In addition, before the reaction gas in the methanation reaction part 30 is supplied to the reaction gas analysis part 90, the catalyst is separated from the reaction gas through the catalyst recirculation part 82. The separated catalyst is supplied to the methanation reaction part 30 again and utilized for the methanation reaction. This can improve the energy efficiency.

While the embodiments of the present disclosure have been described above as specific examples, these embodiments are nothing more than examples. The present disclosure is not limited thereto, and should be construed as having the widest scope in accordance with the basic idea disclosed herein. Those skilled in the art may combine or substitute the disclosed embodiments to implement a pattern of a shape not indicated herein. This also does not depart from the scope of the present disclosure. In addition, those skilled in the art may easily change or modify the disclosed embodiments based on the subject specification. It is apparent that such changes or modifications also belong to the scope of the present disclosure.

What is claimed is:

1. A methane production system, comprising:
a raw material gas supply part configured to store and supply a raw material gas;
a catalyst supply part configured to store and supply a catalyst;
a methanation reaction part connected to the raw material gas supply part and the catalyst supply part and configured to generate a reaction gas by performing a methanation reaction using the raw material gas and the catalyst supplied from the raw material gas supply part and the catalyst supply part;
a temperature measurement part connected to the methanation reaction part and configured to measure a temperature of the methanation reaction part;
a temperature maintaining part connected to the raw material gas supply part and configured to maintain the temperature of the methanation reaction part at a predetermined temperature based on the temperature of the methanation reaction part measured by the temperature measurement part; and
a raw material gas injection part connected to the raw material gas supply part to receive the raw material gas from the raw material gas supply part and configured to inject the raw material gas into the methanation reaction part at a flow rate higher than a flow rate at which the raw material gas is supplied from the raw material gas supply part.

2. The system of claim 1, wherein the temperature maintaining part includes a cooling medium supply part configured to store and supply a cooling medium, and a heat exchanger connected to the cooling medium supply part and configured to exchange heat between the cooling medium supplied from the cooling medium supply part and the reaction heat generated through the methanation reaction, and
the temperature of the cooling medium supplied from the cooling medium supply part to the heat exchanger is determined according to the temperature of the methanation reaction part measured by the temperature measurement part.

3. The system of claim 2, wherein the methanation reaction part includes a reaction body configured to provide a space in which the methanation reaction between the raw material gas and the catalyst is performed, and
the heat exchanger is provided inside the reaction body and installed to be spaced apart from an inner surface of the reaction body.

4. The system of claim 3, wherein an upper flow path through which the raw material gas flows upward is formed in the heat exchanger, and a lower flow path through which the raw material gas flows downward is formed between the inner surface of the reaction body and the heat exchanger.

5. The system of claim 4, further comprising:
a changing part provided inside the reaction body and arranged above the heat exchanger so that the raw material gas flowing upward along the upper flow path enters the lower flow path after colliding with the changing part.

6. The system of claim 5, wherein an upper end of the raw material gas injection part is disposed at a higher position than a lower end of the heat exchanger so that the raw material gas flowing along the lower flow path enters the upper flow path due to a pressure change between the upper end of the raw material gas injection part and the lower end of the heat exchanger.

7. The system of claim 3, wherein the methanation reaction part further includes a catalyst supply port connected at an end to the reaction body and configured to receive the catalyst from the catalyst supply part, a catalyst discharge port connected at an end to the reaction body and configured to discharge the catalyst used in the methanation reaction, a cooling medium supply port having one end connected to the heat exchanger and the other end protruding outward from the reaction body, and configured to receive the cooling medium from the cooling medium supply part, and a cooling medium recovery port having one end connected to the heat exchanger and the other end protruding outward from the reaction body, and configured to recover the cooling medium, which has absorbed the reaction heat generated through the methanation reaction, to the cooling medium supply part.

8. The system of claim 7, further comprising:
a reaction gas processing part connected to the methanation reaction part and configured to post-process the reaction gas supplied from the methanation reaction part;
a reaction gas analysis part connected to the reaction gas processing part and configured to analyze the post-processed reaction gas received from the reaction gas processing part; and
a reaction gas recirculation part connected between the reaction gas analysis part and the raw material gas supply part, and configured to supply at least a part of the post-processed reaction gas to the raw material gas supply part according to the analysis value of the reaction gas analysis part.

9. The system of claim 8, further comprising:
a catalyst discharge part connected to the methanation reaction part,
wherein the catalyst discharge part is configured to discharge the catalyst from the methanation reaction part according to the supply amount of the catalyst supplied to the methanation reaction part through the catalyst supply part.

10. The system of claim 9, further comprising:
a control part configured to control at least one of the raw material gas supply part, the catalyst supply part, the methanation reaction part, the temperature measurement part, the temperature maintaining part, the raw material gas injection part, the changing part, the reaction gas processing part, the reaction gas analysis part, the reaction gas recirculation part and the catalyst discharge part.

11. The system of claim 10, wherein the temperature maintaining part further includes a cooling medium pressure regulation line connected to the cooling medium supply part, a cooling medium pressure regulation valve installed in the cooling medium pressure regulation line, and a cooling medium recovery line arranged to connect the cooling medium recovery port and the cooling medium supply part, and configured to recover the cooling medium, which has absorbed the reaction heat of the methanation reaction, to the cooling medium supply part, and
the control part is configured to control the temperature of the cooling medium supplied to the heat exchanger by controlling the opening and closing of the cooling medium pressure regulation valve according to the temperature of the methanation reaction part measured by the temperature measurement part.

12. The system of claim 10, wherein the catalyst supply part includes a catalyst storage tank configured to store the catalyst, a catalyst supply line arranged to connect the catalyst storage tank and the catalyst supply port and configured to supply the catalyst to the reaction body, and a catalyst supply valve installed in the catalyst supply line, and
the control part is configured to control to adjust the amount of catalyst supplied to the reaction body by controlling the opening and closing of the catalyst supply valve according to the analysis value of the reaction gas analyzed by the reaction gas analysis part.

13. The system of claim 12, wherein the catalyst discharge part includes a catalyst discharge line connected to the catalyst discharge port, and a catalyst discharge valve installed in the catalyst discharge line, and
the control part is configured to adjust the amount of catalyst discharged from the reaction body by controlling the opening and closing of the catalyst discharge valve according to the amount of catalyst supplied to the reaction body by the catalyst supply part.

14. The system of claim 10, wherein the reaction gas processing part includes a reaction gas discharge line connected to the reaction body, a catalyst recirculation part connected to the reaction gas discharge line and configured to receive the reaction gas from the reaction body, separate the catalyst contained in the reaction gas and supply the separated catalyst to the reaction body, a cooler connected to the catalyst recirculation part and configured to cool the catalyst-separated reaction gas received from the catalyst recirculation part, a first connection line configured to connect the catalyst recirculation part and the cooler, a gas-liquid separator configured to separate the reaction gas received from the cooler into a gas product and a liquid product, and a second connection line configured to connect the cooler and the gas-liquid separator.

15. The system of claim 14, wherein the methanation reaction part further includes a catalyst re-supply port connected at an end to the reaction body and configured to receive the catalyst separated through the catalyst recirculation part.

16. The system of claim 15, wherein the catalyst recirculation part includes a dust collector connected to the reaction gas discharge line and configured to separate the catalyst contained in the reaction gas, and a catalyst recirculation line configured to connect the dust collector and the catalyst re-supply port.

17. The system of claim 16, wherein the reaction gas processing part further includes a first discharge line connected to the gas-liquid separator and configured to discharge one of the gas product and the liquid product separated through the gas-liquid separator, a second discharge line connected to the gas-liquid separator and configured to discharge the other of the gas product and the liquid product separated through the gas-liquid separator, and a process pressure regulation valve installed in the first discharge line and provided behind the reaction gas analysis part.

18. The system of claim 17, wherein the reaction gas analysis part is installed in the first discharge line and configured to analyze the composition of the gas product separated through the gas-liquid separator.

19. The system of claim 18, wherein the reaction gas recirculation part includes a first reaction gas recirculation line branched from at least one of the first discharge line and the second discharge line, a compressor installed in the first reaction gas recirculation line, a reaction gas recirculation valve installed in the first reaction gas recirculation line and disposed in front of the compressor, and a second reaction gas recirculation line connected between the compressor and the raw material gas supply part, and
the control part is configured to supply at least a part of the gas product separated through the gas-liquid separator to the raw material gas supply part by controlling the opening and closing of the reaction gas recirculation valve according to the analysis value of the reaction gas analyzed by the reaction gas analysis part.
